(19) United States
(12) Patent Application Publication
(10) Pub. No.: US 2022/0072272 A1
(43) Pub. Date: Mar. 10, 2022

(54) CATHETER INCLUDING A BAMBOO STRUCTURAL SUPPORT MEMBER

(71) Applicant: Covidien LP, Mansfield, MA (US)

(72) Inventors: Edwin Wang, Tustin, CA (US); Ujwal Jalgaonkar, Irvine, CA (US); Xiaodong Ma, Acton, MA (US)

(21) Appl. No.: 17/014,140

(22) Filed: Sep. 8, 2020

(51) Int. Cl. A61M 25/00; A61M 25/01; A61L 29/00; A61L 29/04; A61L 29/18; A61L 29/06

(52) U.S. Cl. CPC A61M 25/0053; A61M 25/0045; A61M 25/0108; A61B 2017/22079; A61L 29/049; A61L 29/18; A61L 29/06; A61L 29/005

(57) ABSTRACT

In some examples, a catheter includes a structural support member including bamboo. For example, a catheter may include a bamboo coil and/or a bamboo braided member positioned between an inner liner and an outer jacket. The structural support member comprising bamboo may be configured to be biodegradable.

### TECHNICAL FIELD

This disclosure relates to a medical catheter.

### BACKGROUND

A medical catheter defining at least one lumen has been proposed for use with various medical procedures. For example, in some cases, a medical catheter may be used to access and treat defects in blood vessels, such as, but not limited to, lesions or occlusions in blood vessels.

### SUMMARY

This disclosure describes medical catheters that include a structural support member including bamboo. In some examples, a catheter includes a bamboo coil and/or a bamboo braided member positioned between an inner liner and an outer jacket. In some examples, the structural support member comprising bamboo may be positioned in one or more of a proximal portion, a medial portion, or a distal portion of a catheter. The structural support member comprising bamboo may be configured to be biodegradable.

In some examples, a catheter may also include a structural support member formed from other biodegradable materials including, but not limited to, silk. For example, a proximal portion of the elongated body can include a first structural support member including bamboo, and at least one of a medial or a distal portion of the elongated body can include a second structural support member including a biodegradable material different from bamboo.

A catheter including a biodegradable structural support member may enable a catheter to be devoid any metal structural support member and enable the catheter to be Magnetic Resonance Imaging (MM) compatible. Eliminating metal structural support members can also facilitate configuring the catheter to be more environmentally friendly because it may have more structures that are biodegradable compared to a catheter including a metal structural support member.

This disclosure also describes examples of methods of forming the catheters described herein and methods of using the catheters.

The following clauses provide some examples of the disclosure:

Clause 1: A catheter includes an elongated body includes an inner liner; a structural support member comprising bamboo; and an outer jacket, wherein the structural support member is positioned between the inner liner and the outer jacket.

Clause 2: The catheter of clause 1, wherein the structural support member comprises a bamboo coil.

Clause 3: The catheter of clause 1 or clause 2, wherein the structural support member comprises a braid comprising one or more bamboo fibers or a combination of bamboo and silk fibers.

Clause 4: The catheter of any of clauses 1-3, wherein the structural support member comprises one or more axially extending bamboo stiffening elements.

Clause 5: The catheter of any of clauses 1-4, wherein at least one of the outer jacket or the inner liner comprises a biodegradable polymer.

Clause 6: The catheter of any of clauses 1-5, wherein the inner liner, the structural support member, and the outer jacket are laminated together.

Clause 7: The catheter of any of clauses 1-6, wherein the elongated body comprises: a proximal portion comprising the inner liner, the structural support member comprising bamboo, and the outer jacket; a medial portion comprising the inner liner, the structural support member comprising bamboo, and the outer jacket; and a distal portion comprising the inner liner and the outer jacket, the medial portion being positioned between the proximal portion and the distal portion.

Clause 8: The catheter of clause 7, wherein the structural support member comprises a first structural support member, and wherein the distal portion comprises a second structural support member comprising a material different from the first structural support member.

Clause 9: The catheter of clause 7, wherein a section of the outer jacket in at least the proximal portion comprises at least one of polyglycolide (PGLA), polylactide (PLA), or Beta-calcium meta phosphate fiber.

Clause 10: The catheter of clause 7, wherein a section of the outer jacket in at least the distal portion comprises polybutylene adipate terephthalate (PBAT).

Clause 11: The catheter of any of clauses 1-10, wherein the structural support member comprises a first structural support member, and wherein the elongated body comprises: a proximal portion comprising the inner liner, the first structural support member, and the outer jacket; a medial portion comprising the inner liner and the outer jacket; and a distal portion comprising the inner liner and the outer jacket, the medial portion being positioned between the proximal portion and the distal portion, wherein at least one of the medial portion or the distal portion comprises a second structural support member including a biodegradable material different from bamboo.

Clause 12: The catheter of clause 11, wherein the biodegradable material comprises silk.

Clause 13: The catheter of any of clauses 1-12, wherein the inner liner comprises an inner liner proximal portion, an inner liner medial portion, and an inner liner distal portion, the inner liner medial portion being positioned between the inner liner proximal and distal portions, wherein the inner liner proximal portion is formed from a different material than at least one of the inner liner medial portion or the inner liner distal portion.

Clause 14: The catheter of clause 13, wherein the inner liner proximal portion comprises PBAT and the at least one of the inner liner medial portion or the inner liner distal portion comprises polytetrafluorethylene (PTFE).

Clause 15: The catheter of any of clauses 1-14, wherein the elongated body further comprises a radiopaque marker.

Clause 16: The catheter of clause 15, wherein the radiopaque marker comprises PBAT and a radiopaque material.

Clause 17: A catheter includes an elongated body includes an inner liner; a structural support member includes a first structural support member; and a second structural support member; and an outer jacket, comprising a biodegradable polymer, wherein the structural support member comprises bamboo; wherein the structural support member is positioned between the inner liner and the outer jacket; wherein the first structural support member is arranged to be parallel to a longitudinal axis of the elongated body; and wherein the second structural support member is coiled or braided around the inner liner.

Clause 18: The catheter of clause 17, wherein the structural support member comprises one or more axially extending bamboo stiffening elements.

Clause 19: The catheter of clause 17 or clause 18, wherein the inner liner comprises a biodegradable 18 polymer.

Clause 20: The catheter of any of clauses 17-19, wherein the inner liner, the structural support member, and the outer jacket are laminated together.

Clause 21: The catheter of any of clauses 17-20, wherein the elongated body comprises: a proximal portion comprising the inner liner, the structural support member comprising bamboo, and the outer jacket; a medial portion comprising the inner liner, the structural support member comprising bamboo, and the outer jacket; and a distal portion comprising the inner liner and the outer jacket, the medial portion being positioned between the proximal portion and the distal portion.

Clause 22: The catheter of clause 21, wherein a section of the outer jacket in at least the proximal portion comprises at least one of polyglycol-lactoamide (PGLA), PGLA and beta calcium meta phosphate fibers, or polyactide (PLA), or PLA and beta calcium meta phosphate fibers.

Clause 23: The catheter of clause 21, wherein a section of the outer jacket in at least the distal portion comprises PBAT.

Clause 24: The catheter of any of clauses 17-23, wherein the elongated body comprises: a proximal portion comprising the inner liner, the first structural support member, and the outer jacket; a medial portion comprising the inner liner and the outer jacket; and a distal portion comprising the inner liner and the outer jacket, the medial portion being positioned between the proximal portion and the distal portion, wherein at least one of the medial portion of the distal portion comprises the second structural support member; and wherein the second structural support member comprises a biodegradable material different from bamboo.

Clause 25: The catheter of clause 24, wherein the biodegradable material comprises silk.

Clause 26: The catheter of any of clauses 17-25, wherein the inner liner comprises an inner liner proximal portion, an inner liner medial portion, and an inner liner distal portion , the inner liner medial portion being positioned between the inner liner proximal and distal portions, wherein the inner liner proximal portion is formed from a different material than at least one of the inner liner medial portion or the inner liner distal portion.

Clause 27: The catheter of clause 26, wherein the inner liner proximal portion comprises PBAT and the at least one of the inner liner medial portion or the inner liner distal portion comprises PTFE.

Clause 28: The catheter of any of clauses 17-27, wherein the elongated body further comprises a radiopaque marker.

Clause 29: The catheter of clause 28, wherein the radiopaque marker is magnetic resonance imaging compatible.

Clause 30: The catheter of clause 28, wherein the radiopaque marker comprises PBAT and a radiopaque material.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a conceptual side view of an example catheter, which includes an elongated body comprising a structural support member formed at least partially from bamboo.

FIG. 2 is a conceptual cross-sectional view of the elongated body of FIG. 1, where the cross-section is taken along line A-A in FIG. 1 and in a direction orthogonal to a longitudinal axis of the elongated body.

FIG. 3 is a conceptual cross-sectional view of the elongated body of FIG. 1, where the cross-section is taken in a direction parallel to a longitudinal axis of the elongated body.

FIG. 4 is a conceptual cross-sectional view of the proximal portion of the elongated body of FIG. 1, where the cross-section is taken in a direction parallel to a longitudinal axis of the elongated body.

FIGS. 5 and 6 are conceptual cross-sectional views of the medial portion of the elongated body of FIG. 1, where the cross-section is taken in a direction parallel to a longitudinal axis of the elongated body and illustrate different example configurations of a structural support member comprising bamboo.

FIG. 7 is a conceptual cross-sectional view of a distal portion and distal tip of the elongated body of FIG. 1, where the cross-section is taken in a direction parallel to a longitudinal axis of the elongated body.

### DETAILED DESCRIPTION

This disclosure describes a medical catheter that includes a flexible elongated body configured to be navigated through vasculature of a patient. The elongated body includes a structural support member formed at least partially from bamboo (e.g., partially or entirely from bamboo). For example, the elongated body may include one or more axially extending bamboo stiffening elements, one or more bamboo coils, one or more bamboo braided structures, or any combination of the aforementioned structures. The structural support member at least partially formed from bamboo is at least partially biodegradable in some examples. In some examples, the structural support member of the elongated body includes one or more additional biodegradable materials, such as, but are not limited to, silk. The silk may be formed by any suitable insect or other animal, such as, but not limited to, a spider or a silkworm.

A structural support member of a catheter can enhance kink resistance of an elongated body of the catheter, increase column strength of the elongated body, and/or increase burst strength of the elongated body. Thus, a material from which the structural support member is formed should be relatively strong, but also relatively flexible so as to enable the catheter to be navigated through relatively tortuous vasculature in a patient. Bamboo can provide the desired strength and flexibility characteristics to a structural support member. Bamboo possesses properties that may provide one or more advantages when used to form a structural support member of a catheter. For example, bamboo may be naturally antibacterial, antifungal, and/or antistatic. In addition, bamboo can possess at least some physical properties comparable to stainless steel and nitinol, such as a tensile strength equivalent to that of stainless steel while being biodegradable. In some cases, bamboo fibers may be more flexible and more resilient than the nitinol and stainless steel alloy wires used to form structural support members of catheters.

Bamboo may be compatible with thermoplastics and thermoset polymers used to form other parts of the elongated body of a catheter, such that the bamboo and one or more polymer such as polylactide (PLA), polyglycol-lactoamide (PGLA), or combination of PLA, PGLA, and small percentage, 1 to 5% by weight, of Beta-calcium meta phosphate bioinert fibers may be combined to form a composite wall, the composite wall may enable the elongated body to be more stiff with higher column strength and higher pushability and to better transmit a push force to the distal tip of the catheter, even if, for example, the composite wall is only at a proximal portion of the elongated body.

The bamboo fibers described herein may be extracted from the bamboo using any suitable technique, including, but is not limited to, alkali treatment. The tensile strength of the bamboo may be independent of the location of the bamboo fibers in the bamboo.

In some examples, a catheter described herein includes an inner liner, an outer jacket, and the structural support member formed at least partially from bamboo positioned between the inner liner and the outer jacket. In some examples, the structural support member and at least one of the inner liner or the outer jacket are formed from a biodegradable material.

In some examples, an elongated body of a catheter includes a distal portion, a medial portion, and a proximal portion, where the medial portion is positioned between the proximal and the distal portions. The proximal, medial, and distal portions may have different configurations, (e.g., stiffnesses). For example, the distal portion may be more flexible than the proximal and medial portions and the proximal portion may be stiffer than the distal and medial portions. In some examples, the distal, medial, and proximal portions may each comprise a different combination of any the materials disclosed herein. At least the proximal portion includes a structural support member comprising bamboo. Bamboo is relatively stiff compared to other biodegradable structural support member materials (e.g., silk fibers), such that the relatively stiff bamboo may be particularly useful in a proximal portion of the elongated body where axial column strength for pushability is desirable.

For example, a proximal portion of the elongated body may include a structural support member comprising bamboo fibers. In some examples, the bamboo fibers may be axially extending bamboo stiffening elements arranged parallel to a longitudinal axis of the elongated body to provide increased axial strength to the proximal portion. In addition to, or instead of the axially extending bamboo elements, the proximal portion can include a coil and/or braid formed at least partially from bamboo. In some examples, the inner liner of the proximal portion may include polybutylene adipate terephthalate (PBAT) and/or the outer jacket of the proximal portion may include an environmental degradable polymer, such as polycaprolactone (PCL)-based polyurethane (Poly-E-caprolactone-based polyurethane) with 2-dimethyl amino ethyl methacrylate (DEM) as pendant group in polyglycol-lactoamide biodegradable polymers including, but are not limited to, PGLA or PLA. PBAT has a relatively low modulus and stiffness, but high flexibility and toughness, and it may be particularly well suited for blending with other biodegradable polymers, such as PLA.

In some examples, a medial portion of the elongated body distal to the proximal portion includes a structural support member comprising bamboo fibers and/or silk fibers. For example, the structural support member of the medial portion may include a coil and/or braid formed from the bamboo fibers and/or silk fibers. In some examples, the inner liner of the medial portion includes polytetrafluoroethylene (PTFE) and/or the outer jacket of the medial portion includes at least one of a stiff, high flexural-modulus bioplastic and a soft, low flexural-modulus bioplastic. The high flexural-modulus bioplastic may include, for example, PGLA and the low flexural-modulus bioplastic may include, for example, PBAT. Including both the high flexural-modulus bioplastic and the low flexural-modulus bioplastics in the outer jacket of the medial portion may help balance the column strength of the medial portion against the shaft stability of the medial portion.

In some examples, a distal portion of the elongated body distal to the medial portion includes a structural support member comprising silk fibers. For example, the silk fibers may be coiled or braided around the inner liner. In some examples, the inner liner of the distal portion includes PTFE and/or the outer jacket of the distal portion is formed from PBAT. The structural support member, the PTFE inner liner, and the PBAT outer jacket can be connected together (e.g., connected via an adhesive or laminated via application of heat) to define a relatively soft (e.g., compared to the proximal and/or medial portions) longitudinal section of the elongated body. In some examples, the distal portion may be between 1 and 25 centimeters in length and forms a distal-most part of the elongated body, which can also correspond to a distal-most portion of the catheter in some examples.

In some examples, the distal portion may include a polymer radiopaque marker to facilitate fluoroscopic or MM visualization of the distal tip of the catheter. Thus, the radiopaque marker can be MM compatible in some examples. The polymer marker band may be formed from any suitable polymer embedded, doped or compounded with, or otherwise including a radiopaque material. For example, the polymer radiopaque marker can include PBAT and one or more radiopaque materials including, but are not limited to, gadolinium oxide, barium sulfate, tantalum, tungsten, platinum, or other relatively high-density radiopaque materials. Thus, the catheter in these example can be devoid of a solid metal radiopaque marker, better enabling the catheter to be Mill-compatible.

The distal tip of the catheter may define an atraumatic tip that provides a relatively smooth surface for contacting a blood vessel of a patient and may have a hydrophilic coating in some examples. The distal tip may also include biodegradable polymers such as PBAT or PTFE. PBAT is relatively flexible, such that a distal tip formed from PBAT may conform relatively well to a guidewire (introduced in a lumen of the catheter) for tracking of the catheter over the guidewire and navigation of the catheter to a target site within a patient.

In some examples, the inner liner, the structural support member, and the outer jacket layer of the elongated body may be laminated or otherwise connected together (e.g., via an adhesive) to form a composite catheter wall. The lamination may be achieved using any suitable technique, such as through the application of heat, a soft adhesive such as polyurethane (PU), or any combination thereof.

A catheter including a biodegradable structural support member (e.g., formed from bamboo alone or bamboo in combination with silk fiber) may enable a catheter to be devoid any metal structural support member and enable the catheter to be MM compatible. Eliminating metal structural support members can also facilitate configuring the catheter to be more environmentally friendly because it may have more structures that are biodegradable compared to a catheter including a metal structural support member.

In some examples, a catheter described herein may be made primarily of or even entirely of biodegradable materials and may decompose completely over time, leaving little remaining solid waste. By comparison, catheters with metal structural support members (e.g., nitinol or stainless steel) cannot be completely decomposed and will leave more solid waste than the catheter described herein which include a structural support member formed from bamboo or a combination of bamboo and silk fibers.

Bamboo is particularly well suited to be used to form a structural support member of a catheter because of its properties, including its strength, antibacterial properties, antifungal properties, and/or anti-static properties. In some cases, a catheter comprising a structural support member formed from bamboo may be more flexible and more resilient than a catheter that is otherwise constructed the same except for the inclusion of a metal structural support member because bamboo fibers may be more flexible and more resilient than the metal wires used to form a metal structural support member. In addition, a catheter including a structural support member formed from bamboo may help minimize the amount of metal in the catheter, thereby enabling the catheter to be relatively more light weight compared to catheters including a metal structural support member, while maintaining similar tensile strengths as a result of the physical properties of bamboo fibers.

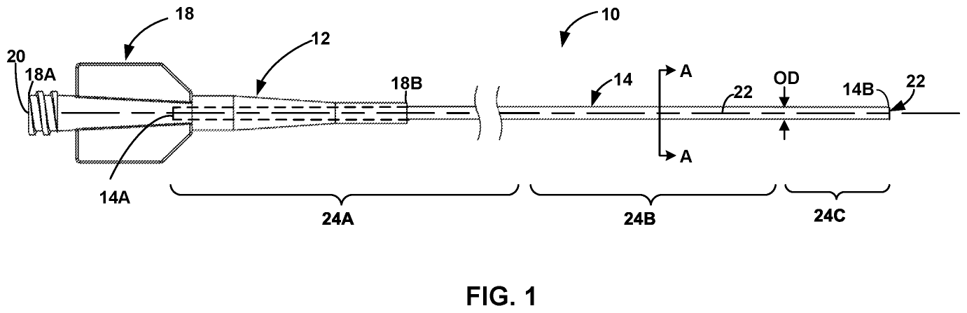

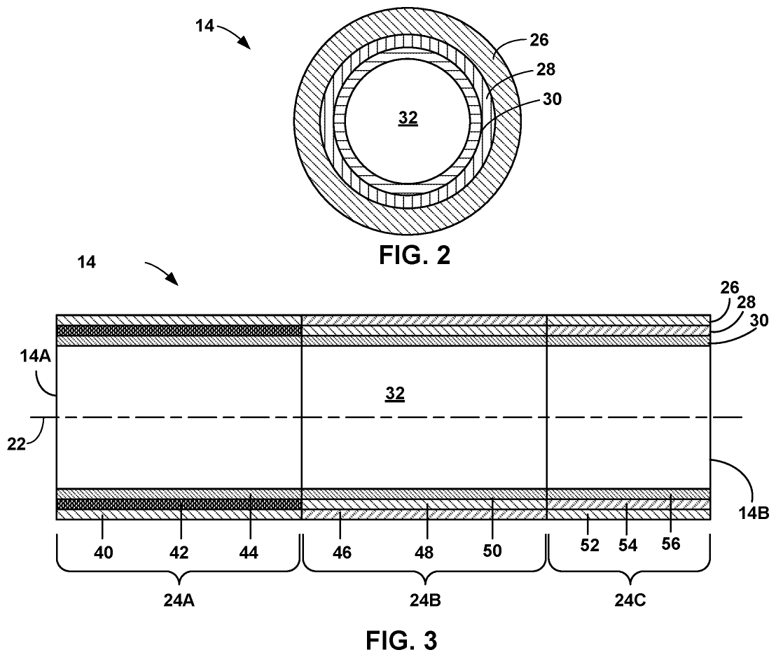

FIG. 1 is a conceptual side view of an example catheter 10, which includes an elongated body 14 comprising a structural support member formed at least partially from bamboo. Catheter 10 further includes a hub 18 positioned at a proximal end 14A of elongated body 14. Elongated body 14 extends from proximal end 14A to distal end 14B, and defines a proximal portion 24A, medial portion 24B, and distal portion 24C. Elongated body 14 may define at least one inner lumen 32 (shown in FIG. 2) that extends the length of elongated body 14. In the example shown in FIG. 1, proximal end 14A of elongated body 14 is received within hub 18 and can be mechanically connected to hub 18 via an adhesive, welding, or another suitable technique or combination of techniques. Opening 20 defined by hub 18 and located at proximal end 18A of hub 18 is aligned with the inner lumen 32 (shown in FIG. 2) of elongated body 14, such that inner lumen 32 of elongated body 14 may be accessed via opening 20. In some examples, catheter 10 may include a strain relief body 12, which may be a part of hub 18 or may be separate from hub 18.

In other examples, the proximal end of catheter 10 can include another structure in addition to or instead of hub 18. In some examples, catheter hub 18 may include one or more Luer connectors or other mechanisms for establishing connections between catheter 10 and other devices.

Inner lumen 32 of elongated body 14 is configured to receive or deliver one or more medical devices, therapeutic agents, etc. to a distal tissue site, remove thrombus (e.g., by aspiration) from the patient's vasculature, and the like or any combination thereof. In examples in which inner lumen 32 defined by elongated body 14 is used to remove thrombus from vasculature, catheter 10 may be referred to as an aspiration catheter. A suction force (e.g., a vacuum) may be applied to a proximal end of catheter 10 (e.g., opening 20) to draw a thrombus into the inner lumen 32. Accordingly, an aspiration system can comprise an aspiration pump connected to the proximal end of catheter 10 (either directly or via intervening tubing or other component(s)).

In some examples, catheter 10 may be advanced to a target location within vasculature of the patient in cooperation with a guide member (not shown) such as a guidewire, an inner catheter, both a guidewire and an inner catheter, or the like, which may aid in the navigation (e.g., steering and manipulation) or elongated body 14 through the vasculature. For example, inner lumen 32 of elongated body 14 may be configured to receive a guide member or an inner catheter, such that the catheter body may be guided through vasculature over the guide member or the inner catheter.

In some examples, elongated body 14 may be used to access relatively distal vasculature locations in a patient, such as the middle cerebral artery (MCA) in a brain of a patient. The MCA, as well as other vasculature in the brain or other relatively distal tissue sites (e.g., relative to the vasculature access point), may be relatively difficult to reach with a catheter, due at least in part to the tortuous pathway (e.g., comprising relatively sharp twists and/or turns) through the vasculature to reach these tissue sites. A structural support member comprising bamboo alone or in combination with outer biodegradable materials may facilitate access to the relatively distal tissue sites by configuring elongated body 14 to be pushable and flexible. For example, one or more bamboo structural support members arranged axially along elongated body 14 (e.g., along longitudinal axis 22) as axially extending bamboo strengthening elements may strengthen elongated body 14 and improve pushability of elongated body 14. In other examples silk structural support members arranged in elongated body 14 may add both structural integrity to elongated body 14 while enabling elongated body 14 to be relatively flexible to aid navigation to relative distal tissue sites.

In some examples, elongated body 14 may define a consistent outer diameter (OD) or an outer diameter taper (e.g., gradient, gradation, segmented gradient of gradation, or the like). The outer diameter taper may assist with the navigability and/or maneuverability of elongated body 14 through the vasculature of a patient. In some examples, the outer diameter taper may define a continuous transition gradient from an outer diameter of elongated body 14 defined at hub distal end 18B by the outer diameter at distal end 14B of elongated body 14. In other examples, the outer diameter of elongated body 14 may define a discontinuous transition (e.g., a gradation or discrete step-downs) in outer diameter may define a discontinuous transition (e.g., a gradation or discrete step-downs) in outer diameter to define the outer diameter taper.

In some examples, the structural support member of elongated body 14 enables the outer diameter of elongated body 14 to remain relatively small to facilitate distal flexibility while still retaining sufficient strength and pushability at proximal portion 24A. For example, the structural support member along proximal portion 24A may comprise bamboo for pushability and the structural support member along distal portion 24C may comprise silk fibers (e.g., and not bamboo) coiled around an inner liner (not shown in FIG. 1) of elongated body 14 to provide increased flexibility at distal portion 24C. The increased flexibility may provide catheter 10 with improve navigability and maneuverability.

In some examples the working length of elongated body 14 may be measured from hub distal end 18B of hub 18 (marked by the distal end of optional strain relief body 12) to distal end 14B of distal portion 24C. The working length of catheter 10 may depend on the location of the target tissue and/or the medical procedure for which catheter 10 is used. For examples, if catheter 10 is a distal access catheter used to access vasculature in a brain of a patient from a femoral artery access point at the groin of the patient, catheter 10 may have a working length of about 129 centimeters (cm) to about 135 cm, such as about 132 cm, although other lengths may be used. In other examples, or for other applications, the working length of elongated body 14 may have different lengths.

Proximal, medial, and distal portions 24A-24C of elongated body 14 are respective longitudinal sections of elongated body 14. In some examples, combined lengths of proximal, medial, and distal portions 24A-24C of elongated body 14 is equal to the working length of elongated body 14. Proximal, medial, and distal portions 24A-24C of elongated body 14 may each have any suitable length. The length may be measured along longitudinal axis 22 of elongated body 14. In some examples, at least two of the proximal, medial, and distal portions 24A-24C have the same length. In other examples, at least two of the proximal, medial, and distal portions 24A-24C may have different lengths.

In some examples, proximal portion 24A has a length of about 50 cm to about 100 cm, such as about 60 cm to about 80 cm, such as about 75 cm; medial portion 24B has a length of about 20 cm to about 40 cm, such as about 25 cm; and distal portion 24C has a length of about 1 cm to about 25 cm, such as about 2.5 cm to about 10 cm, or about 2.5 cm to about 5 cm. For example, proximal portion 24A may have a length of 70 cm, medial portion 24B has a length of 25 cm, and distal portion 24C has a length of 13 cm, and catheter further comprises a distal tip member and a distal radiopaque marker (e.g., described with reference to FIG. 7) that has a combined length of about 2 mm.

The term “about” as used herein with dimensions may refer to the exact value of the such as when used to describe numerical values, “about” or “approximately” refers to a range within the numerical value resulting from manufacturing tolerances and/or within 1%, 5%, or 10% of the numerical value. For example, a length of about 10 mm refers to a length of 10 mm to the extent permitted by manufacturing tolerances, or a length of 10 mm +/−0.1 mm, +/−0.5 mm, or +/−1 mm in various examples.

In some examples, elongated body 14 (e.g., medial portion 24B and/or distal portion 24C) can be relatively thin-walled, such that it defines a relatively large inner diameter for a given outer diameter, which may further contribute to the flexibility, maneuverability, and navigability of elongated body 14. In some examples, elongated body 14 including a structural support member at least partially formed from bamboo may impart the desirable flexibility and maneuverability to thin-walled regions of elongated body 14.

In some examples, at least a portion of an outer surface of elongated body 14 includes one or more coatings, such as, but not limited to, an anti-thrombogenic coating, which may help reduce the formation of thrombi in vitro, an anti-microbial coating, and/or a lubricating coating. The lubricating coating can be, for example, a hydrophilic coating.

Although not shown in FIG. 1, in some examples, elongated body 14 may comprise a hypotube proximal to proximal portion 24A, e.g., a spiral cut hypotube or a hypotube having another cut pattern, which provides elongated body 14 with proximal stiffness that facilitates pushability of elongated body 14.

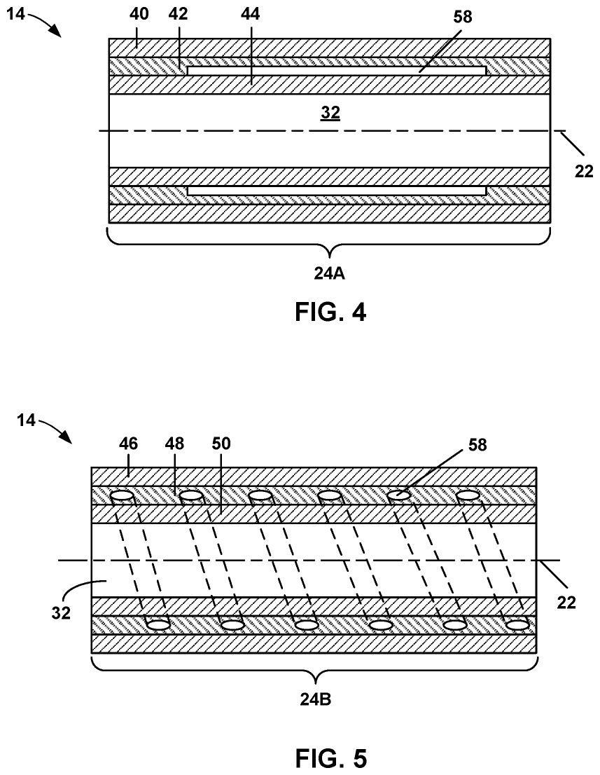

FIG. 2 is a conceptual cross-sectional view of elongated body 14 of FIG. 1, where the cross-section is taken along line A-A in FIG. 1 and in a direction orthogonal to longitudinal axis 22 of elongated body 14. As shown in FIG. 2, in some examples, elongated body 14 includes outer jacket 26, structural support member 28, and inner liner 30, which defines inner lumen 32. Structural support member 28 is positioned between outer jacket 26 and inner liner 30 and comprises one or more biodegradable materials (e.g., bamboo fibers alone or in combination with silk fibers and/or biodegradable polymer fibers). Structural support member 28 is shown schematically in FIG. 2 (and FIG. 3) and may look different in cross-section in other examples, depending on whether structural support member 28 includes one or more axially extending structures, coils, or braids, e.g., as shown with respect to FIGS. 4-6.

In some examples, outer jacket 26 and/or inner liner 30 may also comprise a biodegradable polymer (e.g., biodegradable thermoplastics and/or biodegradable thermoset polymers), which can be the same biodegradable polymer or different biodegradable polymers.

Outer jacket 26 may protect and contain the other components of elongated body 14 of catheter 10 such as structural support member 28 and inner liner 30. Outer jacket 26 may act as a barrier to help prevent liquids such as a patient's body fluids from entering elongated body 14 and to prevent liquids in inner lumen 32 from exiting elongated body 14. In some examples, outer jacket 26 may be a single, continuous component or may be the result of the longitudinal joining and/or lamination of multiple components.

In some examples, outer jacket 26 is formed primarily or entirely of a biodegradable polymer configured to enable outer jacket 26 to decompose over time. In some examples, the biodegradable polymer comprises one or more biodegradable thermoplastics and/or one or more biodegradable thermoset polymers (e.g., PLA, PGLA, and/or PBAT). In some examples, outer jacket 26 may comprise uniformly of one or more of PLA, PGLA, and PBAT. In other examples, portions of outer jacket 26 (e.g., outer jacket proximal portion 40, outer jacket medial portion 46, and/or outer jacket distal portion 52, shown in FIG. 3) may comprise different combinations of biodegradable thermoplastics and/or biodegradable thermoset polymers. For example, outer jacket 46 in proximal portion 24A (outer jacket proximal portion 40) may comprise PGLA or PLA, outer jacket 46 in medial portion 24B (outer jacket medial portion 46) may comprise a mixture of PGLA and PBAT, and outer jacket 46 in distal portion 24C (outer jacket distal portion 52) may comprise PBAT.

In some examples in which outer jacket 26 is formed primarily or entirely of a PLA, PBAT, and/or PGLA fiber, outer jacket 26 may be configured to decompose when exposed to an enzyme that degrades the PLA PBAT, and PGLA fiber into innocuous lactic acid, carbon dioxide, and water matrix. This decomposition may occur from, for example, a time from of 6 months to 2 years. The biodegradation can occur, for example, by diffusion of body fluid, enzyme and water, between the polymer chains. In some examples in which other parts of elongated body 14 are formed from a biodegradable material, such as bamboo and/or silk, these other parts of elongated body 14 can also be configured to degrade in the same or similar time frame and in response to the same one or more enzymes.

In some examples, after elongated body 14 is formed, outer jacket 26 is configured to move longitudinally or radially relative to structural support member 28 and/or inner liner 30. In other examples, outer jacket 26, structural support member 28, and/or inner liner 30 may be laminated together to form a single component using any suitable method (e.g., through the application of heat or a soft or flexible adhesive (e.g., polyurethane (PU)).

Structural support member 28 is located between outer jacket 26 and inner liner 30 and provides structural integrity to elongated body 14. For example, structural support member 28 can be configured to improve kink resistance, axial column strength, and/or burst strength of elongated body 14. Support member 28 is formed primarily or entirely of biodegradable materials including bamboo fibers alone, or bamboo fibers in addition to one or more of silk fibers, and/or bioinert fibers beta-calcium meta phosphate, and/or biodegradable polymers fibers (e.g., PLA fibers). For example, bamboo fibers may be located along an entire length of structural support member 28 (a length being measured along longitudinal axis 22) or may be located along only a part of a length of structural support member 28. For example, bamboo fibers may only be located in portions of structural support member 28 within proximal portion 24A (i.e., structural support member proximal portion 42, shown in FIG. 3) and, in some examples, also within medial portion 24B (i.e., structural support member medial portion 48, shown in FIG. 3).

In some examples, structural support member 28 is formed from the one or more biodegradable materials throughout elongated body 14, e.g., has the same configuration along an entire length of structural support member 28. For example, structural support member 28 may consist essentially of bamboo fibers (e.g., includes bamboo fibers and other materials that do not materially affect the structural characteristics of structural support member 28. As another example, structural support member 28 may include both bamboo and another biodegradable material, such as silk.

In other examples, structural support member 28 comprises a plurality of distinct structural support members each having different configurations (e.g., different materials). For example, structural support member 28 can include a first structural support member (e.g., structural support member proximal portion 42 and/or structural support member distal portion 54, shown in FIG. 3) and a second structural support member (e.g., structural support member distal portion 54, shown in FIG. 3, and, in some examples, structural support member medial portion 48), wherein the first and second structural support members comprise different biodegradable materials. For example, the first structural support member can include bamboo fibers alone or in combination with silk fibers, and the second structural support member can include silk fibers and no bamboo fibers.

Structural support member 28 may, based on the properties of the biodegradable materials selected, impart different structural properties to elongated body 14. The structural properties include strength, stiffness, pushability, flexibility, navigability, and/or maneuverability of elongated body 14. For example, portions of structural support member 28 comprising bamboo fibers may, based on the properties of bamboo, improve the kink resistance, axial column strength, burst strength, stiffness, and/or pushability of elongated body 14. Portions of structural support member 28 comprising silk fibers and not bamboo fibers may, based on the properties of silk, improve flexibility, navigability, and/or maneuverability of elongated body 14.

Structural support member 28 can include axially extending strengthening elements, a coil, and/or a braid. In some examples, structural support member 28 comprises bamboo fibers arranged axially along longitudinal axis 22 and along at least a part (e.g., proximal portion 24A and/or medial portion 24B) of elongated body 14. In addition to or instead of the axially extending bamboo fibers, structural support member 28 can include silk or bamboo fibers or filaments coiled and/or braided around inner liner 30.

Inner liner 30 defines inner lumen 32 and may be formed from any suitable material. Inner lumen 32 may be sized to receive a medical device (e.g., another catheter, a guidewire, an embolic protection device, a stent, a thrombectomy device, an intrasaccular implant such as a coil, or any combination thereof), a therapeutic agent, or the like. At least the inner surface of inner liner 30 defining inner lumen 32 may be lubricious in some examples in order to facilitate the introduction and passage of a device, a therapeutic agent, or the like, through inner lumen 32. In some examples, inner liner 30 is formed primarily or entirely of biodegradable polymers, such as, biodegradable thermoplastics and/or biodegradable thermoset polymers. In other, inner liner 30 may comprise polytetrafluoroethylene (PTFE). In some examples, portions of inner liner 30 (e.g., inner liner proximal portion 44, inner liner medial portion 50, and/or inner liner distal portion 56, shown in FIG. 3) may comprise the same or different biodegradable polymers. Although one inner lumen 32 is shown in FIGS. 2 and 3, in other examples, elongated body 14 can define multiple inner lumens.

FIG. 3 is a conceptual cross-sectional view of the elongated body of FIG. 1, where the cross-section is taken in a direction parallel to longitudinal axis 22 of elongated body 14. FIG. 3 illustrates proximal portion 24A, medial portion 24B, and distal portion 24C of elongated body 14. In various examples, one or more of outer jacket 26, structural support member 28, or inner liner 32 can have different configurations in two or more of proximal portion 24A, medial portion 24B, and distal portion 24C. To help illustrate and describe this, FIG. 3 illustrates outer jacket 26 including outer jacket proximal portion 40, outer jacket medial portion 46, and outer jacket distal portion 52; structural support member 28 including structural support member proximal portion 42, structural support member medial portion 48, and structural support member distal portion 54; and inner liner 32 including inner liner proximal portion 44 inner liner medial portion 50, and inner liner distal portion 56.

In some examples, two or more of the outer jacket portions 40, 46, 52 have a unitary body constructions and/or two or more of outer jacket portions 40, 46, 52 are physically separate structures that may be directly or indirectly connected together. Similarly in some examples, two or more of the inner liner portions 44, 50, 56 have a unitary body constructions and/or two or more of inner liner portions 44, 50, 56 are physically separate structures that may be directly or indirectly connected together. In some examples, two or more of the structural support member portions 42, 48, 54 have a unitary body constructions and/or two or more of structural support member portions 42, 48, 54 are physically separate structures that may be directly or indirectly connected together.

Proximal portion 24A of elongated body 14 includes outer jacket proximal portion 40, structural support member proximal portion 42, and inner liner proximal portion 44. Proximal portion 24A may be at least partially covered by stress relief body 12 (shown in FIG. 1). Proximal portion 24A may be connected to hub 18 at proximal end 14A (shown in FIG. 1). Medial portion 24B of elongated body 14 comprises outer jacket medial portion 46, structural support member medial portion 48, and inner liner medial portion 50. Distal portion 24C of elongated body 14 comprises outer jacket distal portion 52, structural support member distal portion 54, and inner liner distal portion 56.

In some examples, outer jacket proximal portion 40 may be formed from a biodegradable polymer including biodegradable thermoplastics and/or biodegradable thermoset polymers. In some examples, outer jacket proximal portion 40 comprises PGLA, PLA, or a mixture of both PGLA, Beta-calcium meta phosphate fiber, and PLA. In some examples, outer jacket proximal portion 40 may comprise the same biodegradable thermoplastics and/or biodegradable thermoset polymers as outer jacket medial portion 46 and/or outer jacket distal portion 52. Medial portion outer jacket 46 may comprise biodegradable polymers including biodegradable thermoplastics and/or biodegradable thermoset polymers (e.g., PGLA and/or PBAT). Outer jacket distal portion 52 may comprise biodegradable polymers including biodegradable thermoplastics and/or biodegradable thermoset polymers (e.g., PBAT). In some examples, outer jacket proximal portion 40 is formed from PLA having a hardness of 75 D on the Shore D hardness scale, outer jacket medial portion 46 is formed from PLA and PBAT having a hardness of 63 D on the Shore D hardness scale, and outer jacket distal portion is formed from PBAT having a hardness of 35 D on the Shore D hardness scale.

In other examples, outer jacket proximal portion 40, outer jacket medial portion 46, and/or outer jacket distal portion 52 may comprise different biodegradable thermoplastics and/or biodegradable thermoset polymers. For example, in an example, outer jacket proximal portion 40 comprises PGLA, PLA, or combinations thereof, outer jacket medial portion 46 comprises PGLA and/or PBAT, and outer jacket distal portion 52 comprises PBAT.

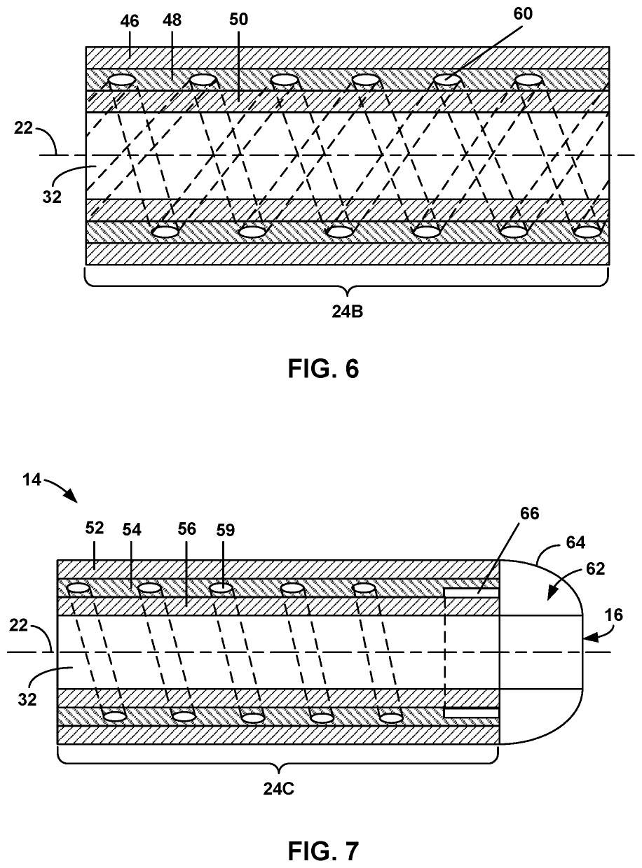

Structural support member proximal portion 42 can be formed at least partially from bamboo alone or bamboo combined with silk fibers and/or other biodegradable material. For example, structural support member proximal portion 42 may comprise bamboo fibers combined with biodegradable polymers such as PLA polymer fibers. In some examples, structural support member proximal portion 42 includes one or more axially extending bamboo elements, as shown in FIG. 4. In examples in which structural support member proximal portion 42 includes a plurality of axially extending bamboo elements, the bamboo elements can be circumferentially evenly or unevenly distributed around inner liner 32. An uneven distribution may provide elongated body 14 with a preferred bending direction. Axially extending bamboo elements may improve axial column strength, burst strength, stiffness, and/or pushability of elongated body 14. In other examples, structural support member proximal portion 42 includes a bamboo coil, as shown in FIG. 4 and/or a bamboo braid, as shown in FIG. 6.

In some examples, structural support member medial portion 48 and/or structural support distal portion 54 are formed from the same biodegradable materials as structural support member proximal portion 42. In other examples, structural support member proximal portion 42, structural support member medial portion 48, and/or structural support member distal portion 52 may comprise different biodegradable materials. For example, structural support member medial portion 48 may comprise silk and not bamboo, or silk and bamboo, and structural support distal portion 54 may comprise silk and not bamboo to provide distal portion 24C of elongated body 14 with increased flexibility relative to proximal portion 24A.

In some examples, inner liner proximal portion 44, inner liner medial portion 50, and inner liner distal portion 56 may be formed from the same material or different materials. Example materials include biodegradable thermoplastics and/or biodegradable thermoset polymers.

FIG. 4 is a conceptual cross-sectional view of an example proximal portion 24A of elongated body 14 of FIG. 1, where the cross-section is taken in a direction parallel to longitudinal axis 22 of the elongated body 14. The cross-sectional view of FIG. 4 illustrates outer jacket proximal portion 40, structural support member proximal portion 42, inner liner proximal portion 44, and inner lumen 32. In the example shown in FIG. 4, structural support member proximal portion 42 include one or more axially extending biodegradable fibers 58, which can be formed from bamboo. Biodegradable fibers 58 extend generally parallel to longitudinal axis 22, e.g., parallel or nearly parallel to longitudinal axis 22 to the extent permitted by manufacturing tolerances. Biodegradable fibers 58 may extend along a full working length of elongated body 14, or along just part of elongated body 14, such as just proximal portion 24A, or proximal and medial portions 24A, 24C, or other combinations of elongated body portions.

In other examples, instead of or in addition to axially extending biodegradable fibers 58, structural support member proximal portion 42 may include one or more coiled biodegradable fibers (e.g., bamboo alone or bamboo with silk or a biodegradable polymer) or one or more braided structures formed from biodegradable fibers. These additional structural support members can be in the same radial layer as biodegradable fibers 58 or can be radially inward or outward (e.g., closer to or further from inner liner proximal portion 44, respectively) from biodegradable fibers 58.

While the cross-section view of FIG. 4 is directed to proximal portion 24A of elongated body, the described structure may also be applied to any other portion of elongated body 14 (e.g., medial portion 24B and/or distal portion 24C) in some examples.

FIG. 5 is a conceptual cross-sectional view of an example medial portion 24B of elongated body 14 of FIG. 1, where the cross-section is taken in a direction parallel to longitudinal axis 22 of elongated body 14. FIG. 5 illustrates an example configuration of medial portion 24B of elongated body 14. The cross-sectional view of FIG. 5 illustrates outer jacket medial portion 46, structural support member medial portion 48 comprising biodegradable coil 59 extending around inner liner medial portion 50, and inner lumen 32.

Biodegradable coil 59 includes one or more biodegradable elements (e.g. bamboo structures, silk structures, PLA polymer fibers, or any combination thereof) defining a coil. Biodegradable coil 56 can have any suitable pitch, e.g., measured as a distance between adjacent turns of the coil. In some examples, a pitch of biodegradable coil 59 is uniform along a length of structural support medial portion 48 and/or other parts of structural support member 28. In some examples, a pitch of biodegradable coil 59 varies along a length of structural support medial portion 48 and/or other parts of structural support member 28, such that a stiffness (or flexibility) varies along the length. In some examples, biodegradable coil 59 may be unidirectional or bidirectional (e.g., cross-wound). In some examples, biodegradable coil 59 may be coiled around inner liner medial portion 50 in a clockwise or counterclockwise direction.

In other examples, structural support member medial portion 48 have another suitable structure. For example, structural support member medial portion 48 may comprise a first structural support layer comprising biodegradable fibers coiled around inner liner medial portion 50 and a second structural support layer comprising biodegradable fibers braided or coiled around the first structural support layer.

FIG. 6 is a conceptual cross-sectional view of another example medial portion 24B of elongated body 14 of FIG. 1, where the cross-section is taken in a direction parallel to longitudinal axis 22 of elongated body 14. The cross-sectional view of FIG. 6 illustrates outer jacket medial portion 46, structural support member medial portion 48 comprising biodegradable braid 60 positioned around inner liner medial portion 50, and inner liner 32.

Biodegradable braid 60 includes one or more elongated biodegradable elements (e.g. bamboo structures, silk structures, PLA polymer fibers, or any combination thereof) defining a braided structure (e.g., woven together to define a braid). Biodegradable braid 60 can have any suitable pic density, where a pic is number of per inch crosses. In some examples, a pic density of biodegradable braid 60 is uniform along a length of structural support medial portion 48 and/or other parts of structural support member 28. In other examples, a pic density of biodegradable braid 60 varies along a length of structural support medial portion 48 and/or other parts of structural support member 28, such that a stiffness (or flexibility) varies along the length.

In other examples, structural support member medial portion 48 have another suitable structure. For example, structural support member medial portion 48 may comprise a first structural support layer comprising biodegradable fibers coiled around inner liner medial portion 50 and a second structural support layer comprising biodegradable fibers braided or coiled around the first structural support layer.

While the cross-section views of FIGS. 5 and 6 are described with reference to medial portion 24B, the described structures herein may also be applied to any other portion of elongated body 14 (e.g., proximal portion 24A and/or distal portion 24C).

FIG. 7 is a conceptual cross-sectional view of distal portion 24C of elongated body 14. In the example shown in FIG. 7, catheter 10 includes a distal tip member 62 at a distal-most portion of elongated body 14. The cross-section is taken in a direction parallel to longitudinal axis 22 of elongated body 14. The cross-sectional view of FIG. 7 illustrates outer jacket distal portion 52, structural support member distal portion 54, inner liner distal portion 56, and inner lumen 32. Structural support member distal portion 54 is shown as including biodegradable coil 59, but structural support member distal portion 54 can include a braided structure (e.g., braid 60 shown in FIG. 6) and/or axially extending fibers (e.g., coil 59 shown in FIG. 5), alone or in combination with coil 59 in other examples.

Distal tip member 62 defines an atraumatic tip of catheter 10. In the example shown in FIG. 7, distal tip member 62 is at a distal-most end of catheter 10, and defines distal-most opening 16 to inner lumen 32. In some examples, distal tip member 62 includes a hydrophilic coating 64, which may reduce surface friction and enhance lubricity of distal tip member 62. In some examples, distal tip member 62 is formed from one or more biodegradable polymers, such as one or more biodegradable thermoplastics and/or biodegradable thermoset polymers (e.g., PBAT and/or PTFE). In some examples, distal tip member 62 may be detachable from distal portion 24C without compromising the structural integrity of elongated body 14.

In some examples, distal tip member 62 is radiopaque or elongated body 14 can include a radiopaque marker 66 to facilitate fluoroscopic or MM visualization of a distal portion of catheter 10. For example, distal tip member 62 and/or radiopaque marker 66 may comprise one or more biodegradable polymers (e.g., PBAT and/or PTFE) and one or more radiopaque materials (e.g., barium sulfate, tantalum, tungsten, platinum, and/or other relatively high-density radiopaque materials). In some examples, radiopaque marker 66 may form a ring or partial ring that extends around the outer perimeter of distal tip member 62, inner liner 14, or distal portion 24C of elongate body 14. In some examples, radiopaque marker 66 may be laminated with inner liner distal portion 56, outer jacket distal portion 52, and/or other parts of distal portion 24C of elongated body 14.

A metallic radiopaque marker (e.g., a platinum band) may contribute to the overall stiffness of a distal tip of a catheter, as well as render the catheter unsuitable for use with MM. Forming the distal tip member 62 and/or band 66 from a radiopaque polymer (a polymer including a radiopaque material distributed therein) may enable the metallic radiopaque marker to be eliminated from the distal tip, thereby enabling the distal portion of catheter 10 to be more flexible. In addition, enabling the metallic radiopaque marker band to be eliminated from catheter 10 without compromising the visibility of catheter 10 under medical imaging may enable distal portion 24C of elongated body 14 and/or the entire elongated body 14 to be formed primarily or entirely of biodegradable materials.

While the cross-sectional view of FIG. 7 includes distal tip member 62, other examples of catheter 10 may not include distal tip member 62 and/or polymeric radiopaque marker 66.

The examples described herein may be combined in any permutation or combination.

Various aspects of the disclosure have been described. These and other aspects are within the scope of the following claims.

What is claimed is:

1. A catheter comprising:
an elongated body comprising:
an inner liner;
a structural support member comprising bamboo; and
an outer jacket, wherein the structural support member is positioned between the inner liner and the outer jacket.

2. The catheter of claim 1, wherein the structural support member comprises a bamboo coil.

3. The catheter of claim 1, wherein the structural support member comprises a braid comprising one or more bamboo fibers or a combination of bamboo and silk fibers.

4. The catheter of claim 1, wherein the structural support member comprises one or more axially extending bamboo stiffening elements.

5. The catheter of claim 1, wherein at least one of the outer jacket or the inner liner comprises a biodegradable polymer.

6. The catheter of claim 1, wherein the elongated body comprises:
a proximal portion comprising the inner liner, the structural support member comprising bamboo, and the outer jacket;
a medial portion comprising the inner liner, the structural support member comprising bamboo, and the outer jacket; and
a distal portion comprising the inner liner and the outer jacket, the medial portion being positioned between the proximal portion and the distal portion.

7. The catheter of claim 6, wherein the structural support member comprises a first structural support member, and wherein the distal portion comprises a second structural support member comprising a material different from the first structural support member.

8. The catheter of claim 1, wherein the structural support member comprises a first structural support member, and wherein the elongated body comprises:
a proximal portion comprising the inner liner, the first structural support member, and the outer jacket;
a medial portion comprising the inner liner and the outer jacket; and
a distal portion comprising the inner liner and the outer jacket, the medial portion being positioned between the proximal portion and the distal portion,
wherein at least one of the medial portion or the distal portion comprises a second structural support member including a biodegradable material different from bamboo.

9. The catheter of claim 8, wherein the biodegradable material comprises silk.

10. The catheter of claim 1, wherein the inner liner comprises an inner liner proximal portion, an inner liner medial portion, and an inner liner distal portion , the inner liner medial portion being positioned between the inner liner proximal and distal portions, wherein the inner liner proximal portion is formed from a different material than at least one of the inner liner medial portion or the inner liner distal portion.

11. The catheter of claim 10, wherein the inner liner proximal portion comprises polybutylene adipate terephthalate (PBAT) and the at least one of the inner liner medial portion or the inner liner distal portion comprises polytetrafluorethylene (PTFE).

12. The catheter of claim 1, wherein the elongated body further comprises a radiopaque marker.

13. The catheter of claim 12, wherein the radiopaque marker comprises polybutylene adipate terephthalate (PBAT) and a radiopaque material.

14. A catheter comprising:
an elongated body comprising:
an inner liner;
a structural support member comprising:
a first structural support member; and
a second structural support member; and
an outer jacket, comprising a biodegradable polymer,
wherein the structural support member comprises bamboo;
wherein the structural support member is positioned between the inner liner and the outer jacket;
wherein the first structural support member is arranged to be parallel to a longitudinal axis of the elongated body; and
wherein the second structural support member is coiled or braided around the inner liner.

15. The catheter of claim 14, wherein the structural support member comprises one or more axially extending bamboo stiffening elements.

16. The catheter of claim 14, wherein the inner liner comprises a biodegradable polymer.

17. The catheter of claim 14, wherein the elongated body comprises:
a proximal portion comprising the inner liner, the structural support member comprising bamboo, and the outer jacket;
a medial portion comprising the inner liner, the structural support member comprising bamboo, and the outer jacket; and
a distal portion comprising the inner liner and the outer jacket, the medial portion being positioned between the proximal portion and the distal portion.

18. The catheter of claim 17, wherein a section of the outer jacket in at least the proximal portion comprises at least one of polyglycol-lactoamide (PGLA), PGLA and beta calcium meta phosphate fibers, or polyactide (PLA), or PLA and beta calcium meta phosphate fibers.

19. The catheter of claim 17, wherein a section of the outer jacket in at least the distal portion comprises polybutylene adipate terephthalate (PBAT).

20. The catheter of claim 14, wherein the elongated body comprises:
a proximal portion comprising the inner liner, the first structural support member, and the outer jacket;
a medial portion comprising the inner liner and the outer jacket; and
a distal portion comprising the inner liner and the outer jacket, the medial portion being positioned between the proximal portion and the distal portion,
wherein at least one of the medial portion of the distal portion comprises the second structural support member; and
wherein the second structural support member comprises a biodegradable material different from bamboo.

21. The catheter of claim 20, wherein the biodegradable material comprises silk.

22. The catheter of claim 14, wherein the elongated body further comprises a radiopaque marker.

23. The catheter of claim 22, wherein the radiopaque marker comprises polybutylene adipate terephthalate (PBAT) and a radiopaque material.
